(19) United States
(12) Patent Application Publication
(10) Pub. No.: US 2017/0349942 A1
(43) Pub. Date: Dec. 7, 2017

(54) MODIFIED OLIGONUCLEOTIDE IMMOBILIZATION ONTO POLYMER SUBSTRATE VIA PHYSISORPTION

(71) Applicant: KONINKLIJKE PHILIPS N.V., EINDHOVEN (NL)

(72) Inventors: STEVEN PAULUS LAMBERTUS KUIJPERS, EINDHOVEN (NL); INGE LAMBERTS, EINDHOVEN (NL); HARMA MARTINE FEITSMA, EINDHOVEN (NL)

(21) Appl. No.: 15/538,225

(22) PCT Filed: Dec. 21, 2015
(86) PCT No.: PCT/EP2015/080813

§ 371 Date: Jun. 21, 2017

Related U.S. Application Data
(60) Provisional application No. 62/104,113, filed on Jan. 16, 2015.

(30) Foreign Application Priority Data
Dec. 31, 2014 (EP) 14200710.3

(51) Int. Cl. C12Q 1/68; B01J 19/00

(52) U.S. Cl. CPC C12Q 1/6837; B01J 19/0046; C12Q 1/6825; B01J 2219/00608; B01J 2219/00576; B01J 2219/0063; B01J 2219/00722; B01J 2219/0061

(57) ABSTRACT

Method for immobilization of a labeled oligonucleotide on a non-modified polymer substrate, the method comprising the following steps: a) providing a mixture comprising liquid, and a labeled oligonucleotide b) applying the mixture of step a) on a non-modified polymer substrate, wherein the oligonucleotide is immobilized on the non-modified polymer substrate via physisorption conveyed by the label of the oligonucleotide and wherein the label for immobilization is covalently bound to the oligonucleotide; and microarrays achieved by this method. The invention further relates to the use of a label attached to an oligonucleotide for immobilization of the labeled oligonucleotide on a non-modified polymer substrate by physisorption. Furthermore the invention relates to the use of the microarrays achieved by the method describe herein for assays and diagnostic kits comprising such microarrays.

### FIELD OF THE INVENTION

Method for immobilization of a labeled oligonucleotide on a non-modified substrate, the method comprising the following steps: a) providing a mixture comprising liquid, and a labeled oligonucleotide b) applying the mixture of step a) on a non-modified polymer substrate, wherein the oligonucleotide is immobilized on the non-modified polymer substrate via physisorption. The invention also relates to microarrays achieved by this method. The invention further relates to the use of a label attached to an oligonucleotide for immobilization of the labeled oligonucleotide on a non-modified polymer substrate by physisorption. Furthermore the invention relates to the use of the microarrays achieved by the method describe herein for assays and diagnostic kits comprising such microarrays.

### BACKGROUND OF THE INVENTION

Biochips or biological microarrays, in particular DNA microarrays, have become an important tool in modern molecular biology and medicine. Typically the chips consist of an arrayed series of a large number of microscopic spots of oligonucleotides, each containing small amounts of a specific nucleic acid sequence. This can be, for example, a short section of a gene or other DNA element that are used as capture probes to hybridize a cDNA or cRNA sample (a target) under conditions, which allow a binding between the capture probe and the corresponding target.

Plastics are often used for the production of disposable substrates, since they are low-cost and can be used for large-scale production. For biochemical assays for the detection of nucleic acids, DNA oligonucleotide probes need to be immobilized onto these substrates. Several methods have been published for immobilization of DNA oligonucleotides onto plastic (i.e. polymer) substrates, such as cyclic olefin copolymer (COC). In most cases, plastic-based microarrays have a modified/functionalized surface to enable the attachment of oligonucleotides. Other immobilization methods describe the binding of (linker-) modified oligonucleotides on non-modified polymer substrates. Brief UV exposure immobilizes DNA oligonucleotides containing a poly(T) and/or poly(C) tag onto various non-modified polymer substrates. (Y. Sun et al, (2012); D. Sabourin et al (2010); N. Kimura, (2006))

Due to the necessary modification of the polymer substrate these methods are costly and time consuming. Further they may have negative effects on the detection process. In particular irradiating nucleic acids with UV light typically leads to damages to the nucleic acid molecule, which may have consequences for the capability of the molecules to hybridize with complementary sequences and could thus compromise their usability in microarray systems. Therefore there is a need for the development of improved methods with decreased costs and production time of microarrays that does not compromise the quality of the assays carried out with these microarrays.

### SUMMARY OF THE INVENTION

The present invention addresses the needs for methods which are less time-consuming and costly for producing microarrays which do not compromise the quality of assays performed with these microarrays.

The above objective is accomplished by a method for immobilization of a labeled oligonucleotide on a non-modified polymer substrate, the method comprising the following steps: a) providing a mixture comprising liquid, and a labeled oligonucleotide, and b) applying the mixture of step a) on a non-modified substrate; wherein the oligonucleotide is immobilized on the non-modified substrate via physisorption.

In a specific embodiment, the polymer substrate is selected from the group consisting of polymethylmethacrylate (PMMA), polycarbonate (PC), polynorbornene, cyclic olefin copolymer (COC), fluorinated polyimide, polystyrene (PS), styrene butadiene copolymer (SBC), acrylonitrile butadiene styrene (ABS), styrene acrilonitrile (SAN) polyethylene (PE), polypropylene (PP) and polysulfone.

In a preferred embodiment the polymer substrate is cyclic olefin copolymer (COC).

In another preferred embodiment the labeled oligonucleotide is DNA, RNA or LNA.

In a further preferred embodiment the labeled oligonucleotide is DNA or LNA.

In a additional preferred embodiment the labeled oligonucleotide is ssDNA or ssLNA.

In another preferred embodiment the labeled oligonucleotide comprises an oligonucleotide to which at least one chemical group is attached, wherein the chemical group is suitable to immobilize the labeled oligonucleotide to the non-modified substrate.

In another embodiment the labeled oligonucleotide comprises an oligonucleotide to which at least one chemical group is attached, wherein the chemical group has preferably a molecular weight from 0.1 to 1000 kDa, from 0.1 to 50 kDa, from 0.1 to 1.5 kDa, from 0.15 to 1 kDa from 0.2 to 0.8 kDa.

In a further embodiment, the chemical group is a chemical group, preferably selected from the group consisting of biotin or sulforhodamine 101 acid chloride (CAS number 82354-19-6; Texas Red).

In a another embodiment the length of the labeled oligonucleotide is from about 2 to about 2000 nucleotides, from about 2 to 500, from about 2 to about 200 nucleotides, from about 2 to about 100, from about 2 to 50 nucleotides.

In a further embodiment the method does not include the step of UV exposure and the oligonucleotide is not covalently bound to the substrate.

In yet another embodiment step b) is followed by the step incubation of the substrate.

In an additional embodiment the above mentioned method steps are followed by the step substrate drying.

In a further embodiment the above mentioned method steps followed by the step washing the substrate, which is optionally followed by the step drying the substrate.

Another aspect of the invention relates to the use of a label attached to an oligonucleotide for immobilization of the labeled oligonucleotide on a non-modified polymer substrate by physisorption.

An additional aspect of the invention refers to a microarray achieved by the method as described above.

A further aspect of the invention relates to a microarray comprising a non-modified polymer substrate and a labeled oligonucleotide which is immobilized on the substrate; wherein there is no covalent bond between the oligonucleotide and the non-modified polymer substrate.

In one embodiment the labeled oligonucleotide further comprises a molecule of interest which is attached to the labeled oligonucleotide.

A further embodiment refers to a microarray comprising a non-modified polymer substrate, a labeled oligonucleotide which is immobilized on the substrate and a oligonucleotide to which a molecule of interest is attached; wherein said oligonucleotide is hybridized to the labeled oligonucleotide immobilized on the polymer substrate and wherein there is no covalent bond between the labeled oligonucleotide immobilized on the substrate and the non-modified polymer substrate.

In one embodiment of the invention the labeled oligonucleotide is immobilized on the non-modified substrate by physisorption.

A further aspect of the invention relates to a diagnostic kit comprising an array of oligonucleotides which are immobilized according to the method as described herein and a control probe containing a defined amount of a known, labeled nucleic acid complementary to a least one of said immobilized oligonucleotides.

An additional aspect of the invention refers to the use of a microarray as defined above for hybridization assays, for surface amplification, for quantitative and/or multiplexed detection of DNA or RNA molecules, for expression analysis, for comparative genomic hybridization, for detection of single nucleotide polymorphism or for genomic selection-based sequencing.

Another aspect of the invention relates to a method for immobilization of a molecule of interest on a non-modified polymer substrate, the method comprising the steps of the method for immobilization of a labeled oligonucleotide as described herein, wherein the labeled oligonucleotide provided in step a) further comprises a molecule of interest which is attached to the oligonucleotide.

Another aspect of the invention relates to a method for immobilization of a molecule of interest on a non-modified polymer substrate, the method comprising the steps of the method for immobilization of a labeled oligonucleotide as described herein and in addition at least the step of:

c) hybridizing a oligonucleotide to which the molecule of interest is attached.

### BRIEF DESCRIPTION OF THE DRAWINGS

FIG. 1. displays the assay result with a COC substrate printed with a spot of non-modified oligonucleotide, displaying no visible spot upon hybridization with target and labeling with nanoparticles.

FIG. 2 shows the result of a COC substrate printed with a spot of TexasRed-modified oligonucleotide, with a clearly visible spot after hybridization.

FIG. 3 shows the result of a COC substrate printed with a spot of biotin-modified oligonucleotide, with a clearly visible spot after hybridization.

FIG. 4 shows a schematic overview of the hybridzation assay of Example 2.

### DETAILED DESCRIPTION OF EMBODIMENTS

Although the present invention will be described with respect to particular embodiments, this description is not to be construed in a limiting sense.

Before describing in detail exemplary embodiments of the present invention, definitions important for understanding the present invention are given.

As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention the term “consisting of” is considered to be a preferred embodiment of the term “comprising of”. If hereinafter a group is defined to comprise at least a certain number of embodiments, this is meant to also encompass a group which preferably consists of these embodiments only.

Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

In case the terms “first”, “second”, “third” or “(a)”, “(b)”, “(c)”, “(d)” etc. relate to steps of a method or use there is no time or time interval coherence between the steps, i.e. the steps may be carried out simultaneously or there may be time intervals of seconds, minutes, hours, days, weeks, months or even years between such steps, unless otherwise indicated in the application as set forth herein below.

It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

One aspect of the invention refers to a method for immobilization of a labeled oligonucleotide on a non-modified polymer substrate, the method comprising the following steps: providing a mixture comprising liquid, and a labeled oligonucleotide, applying the mixture on a non-modified polymer substrate, wherein the oligonucleotide is immobilized on the non-modified polymer substrate via physisorption.

The terms “non-modified” and “not modified” as used herein refer to a polymer substrate that is not modified or functionalized for example by a silane layer with functionalized groups, like polyethyleneglycol, amine, epoxide, isothiocyanate, poly-L-lysine, avidin or streptavidin. That means inter alia that the polymer substrate has not a modified or functionalized surface, such as hydrophobic monolayer formed of alkyl chains or hydrophilic layers formed of polyethylene glycol or oligoethylen glycol chains, epoxy group-containing polymer layer, aminosilanized surface and is not coated with substances that promote the phosphate bonding, such as amines, guadinium groups, amidinium groups, imidazolium group, uncharged H-bond donor such as an aldehyde, alcohol or fomamide or uncharged inorganic H-bond donor such as SiO₂, TiO₂, AlO₂. The term further means that the polymer substrate is for example not contacted with an organic thin film, covering all or some of the surface of the polymer substrate. To the polymer substrate no reactive groups or functional groups are attached. In particular, the terms “non-modified” and “not modified” mean that the binding properties of the substrate are not changed. More particularly, it is meant that no molecules, in particular no molecules enhancing the binding of oligonucleotides, are added to the polymer substrate or that binding groups of the polymer substrate are not activated.

The term “immobilization of a labeled oligonucleotide on a non-modified polymer substrate” relates to the association of labeled oligonucleotides to a non-modified polymer substrate via molecular interactions which impede a detaching of the oligonucleotides, e.g. during washing, rinsing or chemical hybridization steps. In the method of invention such molecular interactions are not based on covalent chemical bonds, but are based on adsorption in particular physisorption between the substrate and the labeled oligonucleotide to be immobilized. The adsorptive force binding the nucleotide to the substrate may be selected from the group consisting of hydrogen bonding, electrostatic interactions, Van der Waals interactions, hydrophobic interactions or combinations thereof.

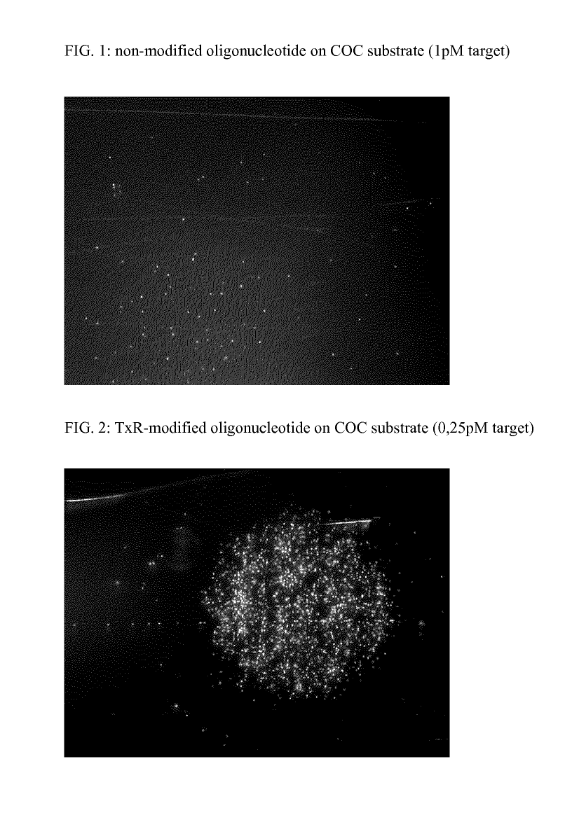

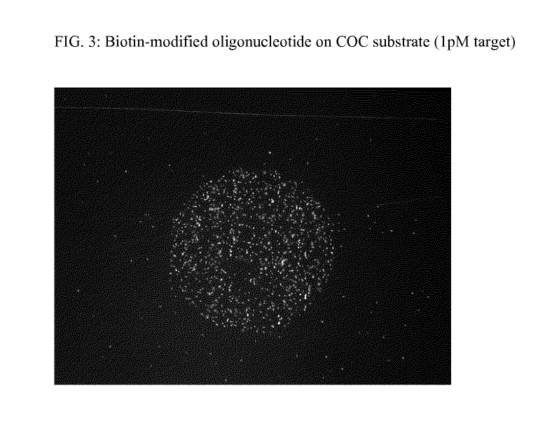

The adsorption between the substrate and the oligonucleotide is conveyed by the label of the labeled oligonucleotide. This becomes clear from FIG. 1 to FIG. 3, which show that the oligonucleotide without a label are not immobilized on the COC substrate (FIG. 1), while when the oligonucleotide is labeled with Texas Red (FIG. 2) or biotin (FIG. 3) a clear spot comprising several labeled oligonucleotides can be detected showing that the labeled oligonucleotide is immobilized on the substrate. This clearly shows that only oligonucleotides with a label as defied herein are immobilized on polymer substrates, such as COC.

Therefore, in the present invention the immobilization of the labeled oligonucleotide is mediated by the adsorption of the label of the labeled oligonucleotide to the non-modified polymer substrate.

A “target” or “target molecule” as used herein may be any suitable molecule, which allows a specific interaction as described herein above. Examples of target molecules are nucleic acids, proteins, peptides, ligands of any form and format, antibodies, antigens, small molecules like organic, inorganic or mixtures of organic and inorganic structures, e.g. carbohydrates or sugars, polymers, entities like cells or cell fragments or cell sub-portions, e.g. bacterial cells, or fragments thereof, eukaryotic cells or fragments thereof, viral particles or viruses, or any derivative or combination of the aforementioned.

The term “applying” as used herein refers to any method of liquid depositing, such as contact printing or non-contact printing. In a preferred embodiment non-contact printing is used to apply the mixture of step a) on a non-modified substrate.

The term “non-contact printing” as used herein refers to any non-contact printing method in the art suitable for printing mixtures comprising oligonucleotides on polymer substrates such as inkjet printing, spotting or dispensing. For non-contact printing a spotter such as sciFLEXARRAYER S11 (Scienion) may be employed.

A specific embodiment of the invention refers to a method for immobilization of a labeled oligonucleotide on a non-modified polymer substrate, the method comprising the following steps: providing a mixture comprising liquid, and a labeled oligonucleotide, non-contact printing the mixture on a non-modified polymer substrate, wherein the oligonucleotide is immobilized on the non-modified polymer substrate via physisorption.

The term “substrate” as used herein refers to any suitable substrate known to the person skilled in the art. The substrate may have any suitable form or format, e.g. it may be flat, curved, e.g. convexly or concavely curved towards, it may be curled or comprise a wavelike format. An array may further comprise magnetic particles comprising capture molecules. The term substrate as used herein may include solid objects, such as solid slides chambers or solid microfluidic devices, as well as coating layers, for example coating layers of slides, coating layers of chambers or coating layers of microfluidic devices. In one embodiment the substrate refers to solid objects. In specific embodiments the substrate refers to solid slides or solid chambers.

In one embodiment of the invention the polymer substrate is selected from the group consisting of polymethylmethacrylate (PMMA), polycarbonate (PC), polynorbornene, cyclic olefin copolymer (COC), fluorinated polyimide, polystyrene (PS), styrene butadiene copolymer (SBC), acrylonitrile butadiene styrene (ABS), styrene acrilonitrile (SAN), polyethylene (PE), polypropylene (PP) and polysulfone.

In a preferred embodiment the polymer substrate is selected from the group consisting of polymethylmethacrylate (PMMA), polycarbonate (PC), polynorbornene, cyclic olefin copolymer (COC), fluorinated polyimide, styrene butadiene copolymer (SBC), acrylonitrile butadiene styrene (ABS), styrene acrilonitrile (SAN), polyethylene (PE), polypropylene (PP) and polysulfone.

In more preferred embodiment the polymer substrate is selected from the group consisting of polynorbornene, cyclic olefin copolymer (COC), fluorinated polyimide, styrene butadiene copolymer (SBC), acrylonitrile butadiene styrene (ABS), styrene acrilonitrile (SAN), polyethylene (PE) and polysulfone. In a even more preferred embodiment of the invention the polymer substrate is cyclic olefin copolymer (COC).

In another preferred embodiment the labeled oligonucleotide comprises an oligonucleotide to which at least one chemical group is attached, wherein the chemical group is suitable to immobilize the labeled oligonucleotide to the non-modified substrate.

The term “attached” refers to covalent and non-covalent binding. Preferably, “attached” means that the label for immobilization is covalently bound to the oligonucleotide. Preferably, “attached” means that the molecule of interest is covalently bound to the oligonucleotide.

“Suitable to immobilize the labeled oligonucleotide” is to be understood as that between the at least one chemical group and the non-modified substrate adsorptive forces, in particular physisorptive forces, are established so that the labeled oligonucleotide is immobilized to the substrate. This can be inter alia tested following the protocol of Examples 1 to 3 using oligonucleotides which are labeled with the chemical group to be tested. The formation of a spot, for example as shown in FIG. 2 and FIG. 3 indicates that the chemical group is suitable to immobilize the labeled oligonucleotide to the substrate. In contrast, the absence of a spot, for example as shown in FIG. 1 indicates that the chemical group is not suitable to immobilize the labeled oligonucleotide to the substrate. Thus, by means of hybridizing probes with magnetic beads to the sample and detecting the beads by frustrated total internal reflection (f-TIR) the suitability of the oligonucleotide immobilized to the substrate can be tested. However, also other probes marked with different labels can be used for detection in combination with a detection method suitable to detect the label of the probe.

In another embodiment the labeled oligonucleotide comprises an oligonucleotide to which at least one chemical group is attached, wherein the chemical group has preferably a molecular weight from 0.1 to 1000 kDa, from 0.1 to 50 kDa, from 0.1 to 1.5 kDa, from 0.15 to 1 kDa from 0.2 to 0.8 kDa.

In a further embodiment, the at least one chemical group is a hydrophobic chemical group. In a specific embodiment, For example, the hydrophobic chemical group may be a hydrophobic chromophore, a long hydrophobic chain, cholesteryl, PEG or biotin.

The term “hydrophobic,” as used herein, refers to a tendency to not dissolve (i.e., associate) readily in water. A moiety may be hydrophobic by preferring to bond or associate with other hydrophobic moieties or molecules, thereby excluding water molecules.

In a preferred embodiment the chemical group is a protein or non-protein organic fluorophore such as cyanine derivatives, naphthalene derivatives, oxadiazole derivatives, anthracene derivatives, pyrene derivatives, oxazine derivatives, acridine derivatives, arylmethine derivatives or tetrapyrrole derivatives, preferably a xanthene derivative, such as fluoresceine or derivatives thereof or rhodamine or derivatives thereof.

In a preferred embodiment the chemical group is rhodamine or derivatives thereof, such as carboxytetramethylrhodamine (TAMRA), tetramethylrhodamine (TMR) and its isothiocyanate derivative (TRITC) and sulforhodamine 101 and sulforhodamine 101 chloride (Texas Red).

In a preferred embodiment the chemical group is selected from the group consisting of biotin or sulforhodamine 101 acid chloride (CAS number 82354-19-6; Texas Red).

The chemical group may be attached at the 3′-end or at the 5′-end of the oligonucleotide.

In a preferred embodiment the labeled oligonucleotide is labeled RNA, labeled LNA or labeled DNA. In an even more preferred embodiment the oligonucleotide is labeled DNA or labeled LNA.

The labeled oligonucleotide may also comprise nucleotides aptamers such as DNA aptamers, RNA aptamers or LNA aptamers.

The oligonucleotide to be immobilized in accordance with a preferred embodiment of the present invention may be DNA, RNA, PNA, CNA, HNA, LNA or ANA. The DNA may be in the form of, e.g. A-DNA, B-DNA or Z-DNA. The RNA may be in the form of, e.g. p-RNA, i.e. pyranosysl-RNA or structurally modified forms like hairpin RNA or a stem-loop RNA.

The term “PNA” relates to a peptide nucleic acid, i.e. an artificially synthesized polymer similar to DNA or RNA which is used in biological research and medical treatments, but which is not known to occur naturally. The PNA backbone is typically composed of repeating N-(2-aminoethyl)-glycine units linked by peptide bonds. The various purine and pyrimidine bases are linked to the backbone by methylene carbonyl bonds. PNAs are generally depicted like peptides, with the N-terminus at the first (left) position and the C-terminus at the right.

The term “CNA” relates to an aminocyclohexylethane acid nucleic acid. Furthermore, the term relates to a cyclopentane nucleic acid, i.e. a nucleic acid molecule comprising for example 2′-deoxycarbaguanosine.

The term “HNA” relates to hexitol nucleic acids, i.e. DNA analogues which are built up from standard nucleobases and a phosphorylated 1,5-anhydrohexitol backbone.

The term “LNA” relates to locked nucleic acids. Typically, a locked nucleic acid is a modified and thus inaccessible RNA nucleotide. The ribose moiety of an LNA nucleotide may be modified with an extra bridge connecting the 2′ and 4′ carbons. Such a bridge locks the ribose in a 3′-endo structural conformation. The locked ribose conformation enhances base stacking and backbone pre-organization. This may significantly increase the thermal stability, i.e. melting temperature of the oligonucleotide.

The term “ANA” relates to arabinoic nucleic acids or derivatives thereof. A preferred ANA derivative in the context of the present invention is a 2′-deoxy-2′-fluoro-beta-D-arabinonucleoside (2′F-ANA).

In a further preferred embodiment oligonucleotides may comprise a combination of any one of DNA, RNA, PNA, CNA, HNA, LNA and ANA. Particularly preferred are mixtures of LNA nucleotides with DNA or RNA bases.

In another preferred embodiment the oligonucleotides as defined herein above may be single-stranded, double-stranded or may be composed of combinations of single-stranded and double-stranded stretches. The term “single-stranded nucleic acid” relates to oligonucleotides which comprise a single sugar-phosphate backbone and/or are not organized in a helical form. Preferably these oligonucleotides exhibit no secondary structures or intermolecular associations. The term “double stranded nucleic acid” relates to nucleic acid molecules which comprise two sugar-phosphate backbones. In a preferred embodiment the double-stranded nucleic acids are organized in a double helical form. In a further preferred embodiment double-stranded nucleic acids according to the present invention may be composed of different types of nucleic acid molecules, e.g. of DNA and RNA, DNA and PNA, DNA and CNA, DNA and HNA, DNA and LNA, DNA and ANA, or RNA and CNA, RNA and PNA, RNA and CNA, RNA and HNA, RNA and LNA, RNA and ANA, or PNA and CNA, PNA and HNA, PNA and LNA, PNA and ANA or CNA and HNA, CNA and LNA, CNA and ANA, or HNA and LNA, HNA and ANA, or LNA and ANA. They may alternatively also be composed of combinations of stretches of any of the above mentioned nucleotide variants. Most preferred is single stranded DNA (ssDNA) or single stranded LNA (ssLNA).

The oligonucleotide may have a biding affinity for a target molecule.

The oligonucleotide may comprise a spacer which has no binding affinity for a target molecule. The spacer may be an oligonucleotide or a different polymer for example a carbohydrate chain. The spacer may be an oligonucleotide having a length of for example at least 15 nucleotides, preferably of at least 20 nucleotides or of a least 50 nucleotides. The spacer may be a polymer such as a carbohydrate chain having a length of at least 4 nm, 6 nm or 15 nm. The spacer may be at the end of the oligonucleotide that is labeled with the chemical group suitable to immobilize the labeled oligonucleotide to the substrate and therefore the spacer may be at the end of the oligonucleotide which is in proximity to the substrate.

One specific embodiment refers to a method for immobilization of a labeled oligonucleotide on a non-modified polymer substrate, the method comprising the following steps: providing a mixture comprising liquid, and a labeled oligonucleotide, applying the mixture on a non-modified polymer substrate, wherein the oligonucleotide is immobilized on the non-modified polymer substrate via physisorption and wherein the labeled oligonucleotide is labeled ssDNA.

One specific embodiment refers to a method for immobilization of a labeled oligonucleotide on a non-modified polymer substrate, the method comprising the following steps: providing a mixture comprising liquid, and a labeled oligonucleotide, applying the mixture on a non-modified polymer substrate, wherein the oligonucleotide is immobilized on the non-modified polymer substrate via physisorption and wherein the labeled oligonucleotide is labeled ssLNA.

The oligonucleotides may originate from naturally occurring or artificially prepared nucleic acid molecules. They may comprise coding and/or noncoding regions. According to the invention, oligonucleotides may have a length from about 2 to about 2000 nucleotides, more preferably from about 2 to about 500 nucleotides, or from about 2 to 200 particularly preferably from about 2 to about 100 nucleotides. Also preferred is a length of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides.

In particular, the present invention overcomes the problem that the attachment oligonucleotides in particular of 100 nucleotides or less adsorptive forces are not very effective. Substrates and methods increasing the adsorptive forces between nucleotide and substrate impede the ability of the oligonucleotide to form a duplex or promote non-specific target binding to the microarray. By the use of oligonucleotides to which at least one chemical group is attached that is suitable to bind the oligonucleotides to the polymer substrate by adsorption, in particular physisorption, this problem has been overcome.

The method of the invention does not include the step of photochemical cross linking such as UV exposure. The method of the invention also does not employ chemical cross-linking steps. The method does not comprise modifying the charge of the polymer substrate, so that the surface of the polymer substrate has a specific charge.

In a specific embodiment the mixture applied on the non-modified polymer substrate comprises a liquid and a labeled oligonucleotide. The liquid may be any liquid suitable to dissolve oligonucleotides for application and immobilization on a substrate, such as water or a buffer solution such as PBS, carbonate buffers, acetate buffers and citrate buffers. The buffer solution may comprise for example sucrose, trimethylglycin (betain), trehalose, glycerol, Tween, Sarkosyl, PVA and/or PEG. In a preferred embodiment the buffer comprises sucrose and betain.

In a specific embodiment the liquid may not include cationic detergent-like compounds such as cetyltrimethylammonium bromide (CTAB), 1-ethyl-3-(3-dimethylaminopropyl)carbodiimide hydrochloride (EDC), octyldimethylamine (ODA). In another specific embodiment, the liquid may not include CTAB, EDC or ODA.

The concentration of sucrose may be between 1% and 50%, between 5% and 40%, between 10% and 30%, preferably 20%. The concentration of trimethylglycin may be between 1 mM and 50 mM, between 5 mM and 30 mM, between 10 mM and 20 mM, preferably 15 mM.

In a specific embodiment the buffer comprises PBS, sucrose and trimethylglycin.

A specific embodiment of the invention refers to a method for immobilization of a labeled oligonucleotide on a non-modified polymer substrate, the method comprising the following steps: providing a mixture comprising buffer, and a labeled oligonucleotide, applying the mixture on a non-modified polymer substrate, wherein the oligonucleotide is immobilized on the non-modified polymer substrate via physisorption.

A specific embodiment of the invention refers to a method for immobilization of a labeled oligonucleotide on a non-modified polymer substrate, the method comprising the following steps: providing a mixture comprising buffer, and a labeled oligonucleotide, non-contact printing the mixture on a non-modified polymer substrate, wherein the oligonucleotide is immobilized on the non-modified polymer substrate via physisorption.

Another embodiment of the invention refers to a method for immobilization of a labeled oligonucleotide on a non-modified polymer substrate, the method comprising the following steps: providing a mixture comprising liquid, and a labeled oligonucleotide, applying the mixture on a non-modified polymer substrate, wherein the oligonucleotide is immobilized on the non-modified polymer substrate via physisorption, and wherein the method does not include the step of UV exposure and the oligonucleotide is not covalently bound to the substrate.

Another embodiment of the invention refers to a method for immobilization of a labeled oligonucleotide on a non-modified polymer substrate, the method comprising the following steps: providing a mixture comprising buffer, and a labeled oligonucleotide, non-contact printing the mixture on a non-modified polymer substrate, wherein the oligonucleotide is immobilized on the non-modified polymer substrate via physisorption, and wherein the method does not include the step of UV exposure and the oligonucleotide is not covalently bound to the substrate.

Another embodiment of the invention refers to a method for immobilization of a labeled oligonucleotide on a non-modified polymer substrate, the method comprising the following steps: providing a mixture comprising liquid, and a labeled oligonucleotide, applying the mixture on a non-modified polymer substrate, wherein the oligonucleotide is immobilized on the non-modified polymer substrate via physisorption, wherein the non-modified polymer substrate is COC, wherein the labeled oligonucleotide comprises a chemical group which is biotin or a organic non-protein fluorophore such as sulforhodamine 101 acid chloride (CAS number 82354-19-6; Texas Red) and wherein the method does not include the step of UV exposure and the oligonucleotide is not covalently bound to the substrate.

Another embodiment of the invention refers to a method for immobilization of a labeled oligonucleotide on a non-modified polymer substrate, the method comprising the following steps: providing a mixture comprising buffer, and a labeled oligonucleotide, non-contact printing the mixture on a non-modified polymer substrate, wherein the oligonucleotide is immobilized on the non-modified polymer substrate via physisorption, wherein the non-modified polymer substrate is COC, wherein the labeled oligonucleotide comprises a chemical group which is biotin or a organic non-protein fluorophore such as sulforhodamine 101 acid chloride (CAS number 82354-19-6; Texas Red) and wherein the method does not include the step of UV exposure and the oligonucleotide is not covalently bound to the substrate.

Another embodiment of the invention refers to a method for immobilization of a labeled oligonucleotide on a non-modified polymer substrate, the method comprising the following steps: providing a mixture comprising liquid, and a labeled oligonucleotide, applying the mixture on a non-modified polymer substrate, wherein the oligonucleotide is immobilized on the non-modified polymer substrate via physisorption, wherein the labeled oligonucleotide is ssDNA, wherein the non-modified polymer substrate is COC, wherein the labeled oligonucleotide comprises a chemical group which is biotin or a organic non-protein fluorophore such as sulforhodamine 101 acid chloride (CAS number 82354-19-6; Texas Red) and wherein the method does not include the step of UV exposure and the oligonucleotide is not covalently bound to the substrate.

Another embodiment of the invention refers to a method for immobilization of a labeled oligonucleotide on a non-modified polymer substrate, the method comprising the following steps: providing a mixture comprising buffer, and a labeled oligonucleotide, non-contact printing the mixture on a non-modified polymer substrate, wherein the oligonucleotide is immobilized on the non-modified polymer substrate via physisorption, wherein the labeled oligonucleotide is ssDNA, wherein the non-modified polymer substrate is COC, wherein the labeled oligonucleotide comprises a chemical group which is biotin or a organic non-protein fluorophore such as sulforhodamine 101 acid chloride (CAS number 82354-19-6; Texas Red) and wherein the method does not include the step of UV exposure and the oligonucleotide is not covalently bound to the substrate.

Another embodiment the invention refers to a method for immobilization of a labeled oligonucleotide on a non-modified polymer substrate, the method comprising the following steps: providing a mixture comprising liquid and a labeled oligonucleotide, applying the mixture on a non-modified polymer substrate and incubation of the polymer substrate, wherein the oligonucleotide is immobilized on the non-modified polymer substrate via physisorption.

Incubation of the polymer substrate may occur at room temperature, e.g. 18° C. to 25° C., preferably 20° C. to 22° C. for 30 seconds to 5 hours, for 5 minutes to 4 hours, for 10 minutes to 3 hours for 15 minutes to 1 hour, preferably for 30 minutes.

The incubation may occur with a relative humidity of 10% to 90%, 20% to 80%, 30% to 70%, 40% to 60%, preferably 50%.

Another embodiment the invention refers to a method for immobilization of a labeled oligonucleotide on a non-modified polymer substrate, the method comprising the following steps: providing a mixture comprising liquid and a labeled oligonucleotide, applying the mixture on a non-modified polymer substrate, and substrate drying, wherein the oligonucleotide is immobilized on the non-modified polymer substrate via physisorption.

Another embodiment the invention refers to a method for immobilization of a labeled oligonucleotide on a non-modified polymer substrate, the method comprising the following steps: providing a mixture comprising liquid, and a labeled oligonucleotide, applying the mixture on a non-modified polymer substrate, incubation of the substrate and substrate drying, wherein the oligonucleotide is immobilized on the non-modified polymer substrate via physisorption.

The term “substrate drying” as used herein refers to incubation of the substrate at a temperature of about 25° C. to 70° C., of about 30° C. to 45° C., preferably of about 35° C. to 39° C., more preferably of about 37° C. Thereby the liquid of the mixture evaporates. During this step no cross linking between the labeled nucleic acids and the substrate occurs.

The step substrate drying may be carried out for any suitable period of time known to the person skilled in the art, e.g. 30 min to 36 h, for 1 h to 30 h, for 10 to 22 h. The substrate drying may be carried out by any suitable means known to the person skilled in the art, for example a drying chamber or an oven or an incubator. In addition to the temperature, also other parameters like humidity, aeration or ventilation may be adjusted to suitable values known to the person skilled in the art.

The non-modified substrate may be sterilized for example by conventional steam sterilization (autoclaving) or gamma irradiation. Preferably the non-modified substrate is gamma treated.

Another embodiment the invention refers to a method for immobilization of a labeled oligonucleotide on a non-modified polymer substrate, the method comprising the following steps: providing a mixture comprising liquid, and a labeled oligonucleotide, applying the mixture on a non-modified substrate, incubation of the substrate, substrate drying and washing the substrate, wherein the oligonucleotide is immobilized on the non-modified substrate via physisorption.

Another embodiment the invention refers to a method for immobilization of a labeled oligonucleotide on a non-modified substrate, the method comprising the following steps: providing a mixture comprising liquid, and a labeled oligonucleotide, applying the mixture on a non-modified substrate, substrate drying and washing the substrate, wherein the oligonucleotide is immobilized on the non-modified substrate via physisorption.

Another embodiment the invention refers to a method for immobilization of a labeled oligonucleotide on a non-modified substrate, the method comprising the following steps: providing a mixture comprising liquid, and a labeled oligonucleotide, applying the mixture on a non-modified substrate, incubation of the substrate, and washing the substrate, wherein the oligonucleotide is immobilized on the non-modified substrate via physisorption.

Another embodiment the invention refers to a method for immobilization of a labeled oligonucleotide on a non-modified substrate, the method comprising the following steps: providing a mixture comprising liquid, and a labeled oligonucleotide, applying the mixture on a non-modified substrate and washing the substrate, wherein the oligonucleotide is immobilized on the non-modified substrate via physisorption.

For washing the substrate a washing buffer may be used containing PBS and Tween-20. The buffer may be for example 0.5×PBS and 0.01% Tween20.

The substrates may be placed in a in the washing buffer for 1 second to 1 hour, for 10 seconds to 30 minutes, for 30 seconds to 10 minutes, for 45 seconds to 5 minutes, preferably for 1 minute.

The washing buffer may be removed from the substrate by methods known to the person skilled in the art, such as placing the substrate in a dry cabinet.

Another embodiment the invention refers to a method for immobilization of a labeled oligonucleotide on a non-modified substrate, the method comprising the following steps: providing a mixture comprising liquid, and a labeled oligonucleotide, applying the mixture on a non-modified substrate, incubation of the substrate, substrate drying, washing the substrate and removing the washing buffer from the substrate wherein the oligonucleotide is immobilized on the non-modified substrate via physisorption.

Another embodiment the invention refers to a method for immobilization of a labeled oligonucleotide on a non-modified substrate, the method comprising the following steps: providing a mixture comprising liquid, and a labeled oligonucleotide, applying the mixture on a non-modified substrate, incubation of the substrate, substrate drying, washing the substrate and removing the washing buffer from the substrate wherein the oligonucleotide is immobilized on the non-modified substrate via physisorption.

Another aspect of the invention relates to the use of a label attached to an oligonucleotide for immobilization of the labeled oligonucleotide on a non-modified polymer substrate by physisorption.

The adsorption between the substrate and the oligonucleotide is conveyed by the label of the labeled oligonucleotide. This becomes clear from FIGS. 1 to 3, which show that the oligonucleotide without a label are not immobilized on the COC substrate (FIG. 1), while when the oligonucleotide is labeled with Texas Red (FIG. 2) or biotin (FIG. 3) a clear spot comprising several labeled oligonucleotides can be detected showing that the labeled oligonucleotide is immobilized on the substrate. This clearly shows that only oligonucleotides with a label as defied herein are immobilized on polymer substrates, such as COC.

Therefore, in the present invention the immobilization of the labeled oligonucleotide is mediated by the adsorption of the label of the labeled oligonucleotide to the non-modified polymer substrate.

An additional aspect of the invention refers to a microarray achieved by the method as described herein.

The term “microarray” as used herein refers to an ordered or random array presented for interaction between capture molecules in the array and potential interactors in the surrounding environment, e.g. a medium, a reaction solution, preferably a hybridization solution. A microarray may include any two- or three-dimensional arrangement of addressable regions, preferably a two-dimensional arrangement. A typical microarray may contain multiple spots, features, areas of individual immobilization or areas of individual molecular identity. For example, an array may contain more than 2, 5, 10, 50, 100, 500, 750, 1000, 1500, 3000, 5000, 10,000, 20,000, 40,000, 50,000, 70,000, 100,000, 200,000, 300,000, 400,000, 500,000, 750,000, 800,000, 1,000,000, 1,200,000, 1,500,000, 1,750,000, 2,000,000 or 2,100,000 spots, features, capture molecules or areas of individual immobilization or areas of individual molecular identity. These areas may be comprised in an area of less than about 20 cm², less than about 10 cm², less than about 5 cm², less than about 1 cm², less than about 1 mm², less than about 100 μm². In further embodiments the spot size within a microarray may be in a range of about 1 to 300 μm, e.g. have a size of 10, 50, 100, 150, 200, or 250 μm, or have any suitable value in between the mentioned values. In further embodiments, the probe (oligonucleotide) densities may vary according to necessities. Examples of probe densities are densities between 1000 and 5,000,000 probes per square μm, e.g. probe densities of 2000, 10,000, 50,000, 75,000 or 1,000,000 probes per square μm.

In specific embodiments such spots, features, or areas of individual immobilization may comprise any suitable coverage, e.g. a coverage of 1, 1.5, 2, 3, 4, 5, 6, 7, 8, 9, 10 or more of one or more specific sequences, or genomes or parts of genomes. Thus, a microarray may, for example, comprise 1, 2, 3, 4, 5, 6, 7, 8, 9, 10 or more copies of the same capture molecule. Alternatively or additionally, it may comprise variations of a capture molecule, e.g. mismatch variants, variations with single or multiple nucleotide polymorphisms, different versions of single or multiple nucleotide polymorphisms, capture molecules with different starting or termination sequences, e.g. tags, primer bindings sites, endonuclease recognition sites, however with identical core portions etc.

The herein mentioned microarray may comprise “one or more species” of a oligonucleotide molecule or “one or more species” of a molecule of interest attached to a oligonucleotide molecule, i.e. one or more different types of molecule may be present in the microarray. Alternatively, the term “one or more species” also relates to oligonucleotides of the same category or having the same form or format, e.g. DNA, but which are not identical or similar in their molecular identity, e.g. the sequence. Thus, a reaction zone or a capture zone according to the present invention may comprise different oligonucleotides. If oligonucleotides of a different molecular identity, in particular nucleic acids, are present in a reaction zone or a capture zone according to the present invention, these oligonucleotides may be partially identical or partially similar, i.e. have, in particular in the case of nucleic acids, overlaps in terms of sequence or may have no overlap. Oligonucleotides according to the present invention may, for example, comprise, cover or represent the sequence of any suitable area or percentage of a genome, e.g. between about 0.00001% to about 30% of a genome, such as at least about 0.00001, 0.00005, 0.0001, 0.0005, 0.001, 0.005, 0.01, 0.02, 0.05, 0.06, 0.07, 0.08, 0.09, 0.1, 0.11, 0.12, 0.13, 0.14, 0.15, 0.16, 0.2, 0.3, 0.4, 0.5, 0.75, 0.8, 0.9, 1, 1.5, 2, 3, 4, 5, 10, 15, 20, or 30% of the genome of an organism, preferably of a mammal genome, more preferably of the human genome and/or be complementary to such regions. Such an area or percentage may comprise, for example, a group of about 2 to 5,000 genes, e.g. 2, 3, 4, 5, 6, 7, 8, 9, 10, 15, 20, 25, 30, 50, 100, 150, 200, 350, 500, 750, 1000, 1200, 1500, 2000, 2500, 3000, 4000, 5000 or more than 5000 genes. Such genes may either be localized in adjacent genomic areas o regions or my alternatively be dispersed throughout the genome. Also sub-groupings, combinations, pattern, e.g. pattern derived from expression data etc. of genes are envisaged.

A further aspect of the invention relates to a microarray comprising a non-modified polymer substrate and a labeled oligonucleotide which is immobilized on the substrate; wherein there is no covalent bond between the oligonucleotide and the non-modified polymer substrate.

In one embodiment of the invention the polymer substrate is selected from the group consisting of polymethylmethacrylate (PMMA), polycarbonate (PC), polynorbornene, cyclic olefin copolymer (COC), fluorinated polyimide, polystyrene (PS), styrene butadiene copolymer (SBC), acrylonitrile butadiene styrene (ABS), styrene acrilonitrile (SAN), polyethylene (PE), polypropylene (PP) and polysulfone.

In a preferred embodiment of the invention the polymer substrate is cyclic olefin copolymer (COC).

In another preferred embodiment the labeled oligonucleotide comprises an oligonucleotide to which at least one chemical group is attached, wherein the chemical group is suitable to immobilize the labeled oligonucleotide to the non-modified substrate.

In a specific embodiment the labeled oligonucleotide comprises an oligonucleotide to which at least one chemical group is attached, wherein the chemical group has preferably a molecular weight from 0.1 to 1000 kDa, from 0.1 to 50 kDa, from 0.1 to 1.5 kDa, from 0.15 to 1 kDa from 0.2 to 0.8 kDa.

In a further embodiment, the chemical group is a chemical group, preferably selected from the group consisting of biotin or sulforhodamine 101 acid chloride (CAS number 82354-19-6; Texas Red).

In a another embodiment the length of the labeled oligonucleotide is from about 2 to about 2000, from about 2 to 500, from about 2 to about 200 nucleotides, from about 2 to about 100 nucleotides, from about 2 to about 50 nucleotides.

A specific aspect of the invention relates to a microarray comprising non-modified polymer substrate and a labeled oligonucleotide which is immobilized on the non-modified polymer substrate; wherein there is no covalent bond between the oligonucleotide and the non-modified polymer substrate and wherein the oligonucleotide is immobilized on the non-modified polymer substrate by adsorption, in particular, physisorption.

A specific aspect of the invention relates to a microarray comprising non-modified polymer substrate and a labeled oligonucleotide which is immobilized on the non-modified polymer substrate; wherein there is no covalent bond between the oligonucleotide and the non-modified polymer substrate and wherein the labeled oligonucleotide is immobilized on the non-modified polymer substrate via its label by adsorption, in particular, physisorption.

A specific aspect of the invention relates to a microarray comprising a non-modified COC substrate and a labeled oligonucleotide which is immobilized on the non-modified COC substrate; wherein there is no covalent bond between the oligonucleotide and the non-modified COC substrate.

A specific aspect of the invention relates to a microarray comprising a non-modified COC substrate and a labeled oligonucleotide which is immobilized on the non-modified COC substrate; wherein there is no covalent bond between the oligonucleotide and the non-modified COC substrate and wherein the labeled oligonucleotide is labeled ssDNA.

A specific aspect of the invention relates to a microarray comprising a non-modified COC substrate and a labeled oligonucleotide which is immobilized on the non-modified COC substrate; wherein there is no covalent bond between the oligonucleotide and the non-modified COC substrate and wherein the oligonucleotide is immobilized on the non-modified COC substrate by adsorption, in particular, physisorption.

A specific aspect of the invention relates to a microarray comprising a non-modified COC substrate and a labeled oligonucleotide which is immobilized on the non-modified COC substrate; wherein there is no covalent bond between the oligonucleotide and the non-modified COC substrate and wherein the labeled oligonucleotide is immobilized on the non-modified COC substrate via its label by adsorption, in particular, physisorption.

A specific aspect of the invention relates to a microarray comprising a non-modified COC substrate and a labeled oligonucleotide which is immobilized on the non-modified COC substrate; wherein there is no covalent bond between the oligonucleotide and the non-modified COC substrate and wherein the oligonucleotide is labeled with at least one chemical group which is suitable to immobilize the labeled oligonucleotide to the COC non-modified substrate.

A specific aspect of the invention relates to a microarray comprising a non-modified COC substrate and a labeled oligonucleotide which is immobilized on the non-modified COC substrate; wherein there is no covalent bond between the oligonucleotide and the non-modified COC substrate and wherein the oligonucleotide is labeled with at least one chemical group selected from the group consisting of a chromophore or biotin.

A specific aspect of the invention relates to a microarray comprising a non-modified COC substrate and a labeled oligonucleotide which is immobilized on the non-modified COC substrate; wherein there is no covalent bond between the oligonucleotide and the non-modified COC substrate and wherein the oligonucleotide is labeled with at least one chemical group selected from the group consisting of sulforhodamine 101 acid chloride (CAS number 82354-19-6; Texas Red) or biotin.

A specific aspect of the invention relates to a microarray comprising a non-modified COC substrate and a labeled oligonucleotide which is immobilized on the non-modified COC substrate; wherein there is no covalent bond between the oligonucleotide and the non-modified COC substrate, wherein the oligonucleotide is labeled with at least one chemical group selected from the group consisting of sulforhodamine 101 acid chloride (CAS number 82354-19-6; Texas Red) or biotin and wherein the labeled oligonucleotide is labeled ssDNA.

In a one embodiment of the invention the labeled oligonucleotide is immobilized on the non-modified substrate by physisorption.

A further aspect of the invention relates to a diagnostic kit comprising an array of oligonucleotides which are immobilized according to the method as described herein and a control probe containing a defined amount of a known, labeled nucleic acid complementary to a least one of said immobilized oligonucleotides.

A further aspect of the invention relates to a diagnostic kit comprising an microarray comprising oligonucleotides which are immobilized according to the method as described herein and a control probe containing a defined amount of a known, labeled nucleic acid complementary to a least one of said immobilized oligonucleotides.

The term “control probe containing a defined amount of a known, labeled nucleic acid complementary” relates to a well defined oligonucleotide, which is provided with a label, preferably a fluorescent label, and which is used to calibrate and/or test or quality check at least one of the immobilized oligonucleotides present in the form of an array.

An additional embodiment refers to the use of a microarray as defined above for hybridization assays, for surface amplification, for quantitative and/or multiplexed detection of DNA or RNA molecules, for expression analysis, for comparative genomic hybridization, for detection of single nucleotide polymorphism or for genomic selection-based sequencing.

The microarray of the invention is particularly suitable for nucleic acid detection, such as microarrays, hybridization assays such as sandwich hybridization assay, surface amplification. The substrates of the invention may be used in combination with magnetic beads.

The following examples and figures are provided for illustrative purposes. It is thus understood that the example and figures are not to be construed as limiting. The skilled person in the art will clearly be able to envisage further modifications of the principles laid out herein.

Another aspect of the invention relates to a method for immobilization of a molecule of interest on a non-modified polymer substrate, the method comprising the steps of the method for immobilization of a labeled oligonucleotide as described herein, wherein the labeled oligonucleotide provided in step a) further comprises a molecule of interest which is attached to the oligonucleotide.

The molecule of interest may be attached to the opposite side of the oligonucleotide to which the label for immobilization is attached. That means, if the label for immobilization is attached at the 3′-end of the oligonucleotide the molecule of interest may attached to the 5′-end of the oligonucleotide. Vice versa, if the label for immobilization is attached at the 5′-end of the oligonucleotide the molecule of interest may attached to the 3′-end of the oligonucleotide.

In a specific embodiment, if the molecule of interest is attached to the oligonucleotide to which the label for immobilization is attached, the oligonucleotide may have no binding affinity for a target molecule. In this case the oligonucleotide functions as linker to immobilize the molecule of interest to the polymer substrate and does not participate in the binding of the molecule of interest and the target molecule.

A further aspect of the invention relates to a method for immobilization of a molecule of interest on a non-modified polymer substrate, the method comprising the steps of the method for immobilization of a labeled oligonucleotide as described herein and in addition at least the step of: c) hybridizing a oligonucleotide to which the molecule of interest is attached.

The term “molecule of interest” as used herein refers for example to proteins, peptides or antibodies, small molecules, molecule imprinted polymers, protein aptamers or peptide aptamers. The “molecule of interest” may have a binding affinity for a target molecule.

### EXAMPLES

### Example 1—Immobilization of ssDNA on a COC Substrate

A printbuffer, containing a TexasRed-labeled or biotin-labeled ssDNA oligonucleotide (40 nucleotides) or biotin-labeled, 1×PBS with 20% sucrose and 15 mM Betain, is printed on a gamma treated COC substrate with a Scienion sciFLEXARRAYER S11 inkjet printer in a closed environment at room temperature (21° C.), and with a relative humidity of approximately 50%. After printing, the substrates are kept in this environment for half an hour before they are transferred to an incubator with a temperature of 37° C. for overnight drying. The next day, these substrates are washed gently by placing them upside down in a wash bath containing wash buffer for 1 minute (0.5×PBS with 0.01% Tween20). Excess liquid is removed by gently tapping the substrate on a tissue and the substrate is placed in a dry-cabinet for an hour (room temperature, relative humidity ˜10%). The substrates are now ready for further assembly and are kept in sealed pouches at 4° C., with the addition of silica beads to maintain humidity below 10% to minimize condensation.

### Example 2—Sandwich Hybridization Assay

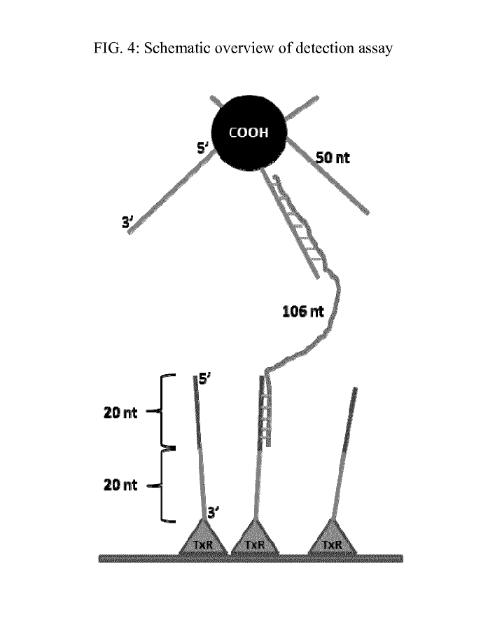

To test the spot quality, a sandwich hybridization assay is performed on the substrate with the immobilized labeled oligonucleotide. Together with magnetic particles labeled with a probe and with a target oligo (solved in a buffer solution), the hybridization is performed. The sandwich hybridization is facilitated by actuating the magnetic particles with magnetic pulses in a buffer solution at a certain temperature (between 40° C. and 60° C.). Non-bound magnetic particles are washed away with a magnet. FIG. 4 shows a schematic cross-section through a substrate with the immobilized TexasRed labeled oligonucleotide to which a magnetic particle is hybridized via a probe. In this specific embodiment, the oligonucleotide comprises a spacer region of 20 nt length at the 3′-end and a region of 20 nt length having a binding activity at its 5′-end for the probe. Thus, the probe is hybridized to the immobilized oligonucleotide and to the magnetic particle.

### Example 3—Detection of Magnetic Particles

The bound magnetic particles are imaged by frustrated total internal reflection (f-TIR) using a CCD camera. A collimated beam of light from a light-emitting diode (λ=625 nm) irradiates the sensor surface in a condition of total internal reflection. A so-called evanescent optical field is generated at the substrate/fluid interface, which decays exponentially and penetrates into the fluid by only a subwavelength distance. In the absence of magnetic particles, no light is extracted from the evanescent optical field (FIG. 1). When magnetic nanoparticles are present at the sensor surface, the particles absorb and scatter part of the light, thus frustrating the total internal reflection and thereby reducing the intensity of the reflected light (FIG. 2, FIG. 3). The substrate surface is imaged onto a CCD camera.

### LIST OF REFERENCE SIGNS

Y. Sun et al, Anal. Bioanal. Chem. (2012) 402:741-748.
D. Sabourin et al, Biomed. Microdevices. (2010) 12:673-681.
N. Kimura, BBRC 347 (2006) 477-484.

1. Method for immobilization of a labeled oligonucleotide on a non-modified polymer substrate, the method comprising the following steps:
a) providing a mixture comprising
liquid, and
a labeled oligonucleotide, and
b) applying the mixture of step a) on a non-modified polymer substrate;
wherein the oligonucleotide is immobilized on the non-modified polymer substrate via physisorption conveyed by the label of the oligonucleotide and wherein the label for immobilization is covalently bound to the oligonucleotide, wherein the polymer substrate is cyclic olefin copolymer.

2. (canceled)

3. The method of claim 1, wherein the labeled oligonucleotide comprises an oligonucleotide to which at least one chemical group is attached, wherein the chemical group is suitable to immobilize the labeled oligonucleotide to the non-modified polymer substrate via physisorption.

4. The method of claim 1, wherein the labeled oligonucleotide comprises an oligonucleotide to which at least one chemical group is attached, wherein the chemical group has preferably a molecular weight from 0.1 to 1000 kDa, from 0.1 to 50 kDa, from 0.1 to 1.5 kDa, from 0.15 to 1 kDa from 0.2 to 0.8 kDa optionally wherein the chemical group is a chemical group, preferably selected from the group consisting of biotin or sulforhodamine 101 acid chloride (CAS number 82354-19-6; Texas Red).

5. (canceled)

6. The method of claim 4, wherein the length of the labeled oligonucleotide is from about 2 to 2,000, from about 2 to 500, from about 2 to about 200 nucleotides, from about 2 to about 100 nucleotides, from about 2 to about 50 nucleotides.

7. The method of claim 1, wherein the method does not include the step of UV exposure and wherein the oligonucleotide is not covalently bound to the substrate.

8. The method of claim 1, wherein step b) is followed by the step incubation of the substrate and/or wherein the method steps are followed by the step substrate drying; and/or wherein the method steps are followed by the step washing the substrate.

9. (canceled)

10. (canceled)

11. The method of claim 1, wherein applying the mixture of step a) on a non-modified polymer substrate is carried out by contact printing or non-contact printing, preferably by non-contact printing.

12. Method for immobilization of a molecule of interest on a non-modified polymer substrate, the method comprising the steps of the method for immobilization of a labeled oligonucleotide of claim 11, wherein the labeled oligonucleotide provided in step a) further comprises a molecule of interest which is attached to the oligonucleotide.

13. Method for immobilization of a molecule of interest on a non-modified polymer substrate, the method comprising the steps of the method for immobilization of a labeled oligonucleotide of claim 11 and in addition at least the step of:
c) hybridizing a oligonucleotide to which the molecule of interest is attached.

14. Use of a label attached to an oligonucleotide for immobilization of the labeled oligonucleotide on a non-modified polymer substrate by physisorption, wherein the polymer substrate is cyclic olefin copolymer.

15. Microarray achieved by the method of claim 13.

16. Microarray comprising
a non-modified polymer substrate and
a labeled oligonucleotide which is immobilized on the polymer substrate via the label of the oligonucleotide;
wherein there is no covalent bond between the oligonucleotide and the non-modified polymer substrate and wherein the label of the oligonucleotide is covalently bound to the oligonucleotide,
wherein the polymer substrate is cyclic olefin copolymer.

17. Microarray of claim 16, wherein the labeled oligonucleotide further comprises a molecule of interest which is attached to the labeled oligonucleotide.

18. Microarray of claim 16 further comprising a oligonucleotide to which a molecule of interest is attached, wherein said oligonucleotide is hybridized to the labeled oligonucleotide immobilized on the polymer substrate.

19. A diagnostic kit comprising a microarray comprising oligonucleotides which are immobilized according to the method of claim 1 and a control probe containing a defined amount of a known, labeled nucleic acid complementary to a least one of said immobilized oligonucleotides.

20. Use of a microarray as defined in claim 18 for hybridization assays, for surface amplification, for quantitative and/or multiplexed detection of DNA or RNA molecules, for expression analysis, for comparative genomic hybridization, for detection of single nucleotide polymorphism or for genomic selection-based sequencing.
